(19) United States
(12) Patent Application Publication
(10) Pub. No.: US 2015/0136160 A1
(43) Pub. Date: May 21, 2015

(54) Oral Stimulatory Product

(71) Applicant: Foundation Brands, LLC, Raleigh, NC (US)

(72) Inventors: Jonathan Fraser Gordon, Wakefield, RI (US); Lea Rubinstein, Wakefield, RI (US)

(73) Assignee: Foundation Brands, LLC, Raleigh, NC (US)

(21) Appl. No.: 14/406,419

(22) PCT Filed: Jun. 7, 2013
(86) PCT No.: PCT/US2013/044758

§ 371 Date: Dec. 8, 2014

Related U.S. Application Data
(60) Provisional application No. 61/657,356, filed on Jun. 8, 2012.

(51) Int. Cl. A24B 15/16; A61K 36/28; A61K 36/752; A61K 36/46; A61K 36/67; A61K 36/77; A24B 15/18; A61K 36/758

(52) U.S. Cl. CPC A24B 15/16; A24B 15/18; A61K 36/28; A61K 36/758; A61K 36/46; A61K 36/67; A61K 36/77; A61K 36/752
USPC 131/359; 131/369

(57) ABSTRACT

The invention provides an oral stimulatory product comprising a stimulating amount of at least one oral stimulant. The oral stimulant being selected from the group consisting of Spilanthes, Szechuan Pepper, roasted Eucommia leaf, and a combination thereof. The product optionally containing Guarana extract, Yerba mate, salt, orange peel, and/or natural flavors.

### TECHNICAL FIELD

The present invention relates to the field of consumable products suitable for use as an oral stimulatory product.

### BACKGROUND

The need for products that provide oral stimulation is increasing. Such products can be useful in the alleviation of symptoms of oral conditions, as well as to replace products that are often considered to be harmful, such as oral tobacco products.

The use of oral tobacco products (chewing tobacco, pouches, etc.) is increasing in the U.S. and internationally as public and private use of smoking tobacco products decreases due to both restrictive legislation and fear of the detrimental health effects of smoking such as lung cancer and emphysema. Unfortunately, oral tobacco products are also strongly associated with a substantial increased risk for oral cancers.

Oral tobacco consumption provides five main effects that are familiar and desirable to the oral tobacco user:
Oral absorption of nicotine providing a stimulant effect;
Oral sensation (burning, tingling, pepperiness, numbing);
Stimulation of salivation and subsequent spitting;
Flavor (tobacco and added flavors such as Wintergreen, Mint etc.);
Oral stimulation (oral fixation) from having a wad of tobacco or a pouch in the gum.

A number of products available on the market attempt to substitute for oral tobacco use. These are variously made from mint leaves, corn silk, red clover, coffee grounds etc. Some of them contain cayenne pepper in an attempt to provide oral sensation. Others contain caffeine or caffeine containing substances such as coffee grounds and Guarana in an attempt to simulate the stimulative effects of nicotine. Some also contain FD&C colors to color the base substance and to try to simulate the color of traditional tobacco “spit.”

None of the alternative products promote salivation and spitting in the manner of oral tobacco products. Neither do they adequately simulate the oral sensation (burning, tingling, pepperiness, numbing) of oral tobacco products. Neither do they simulate the flavor of oral tobacco products. Neither do they simulate the stimulation provided by oral tobacco products.

It appears that the predominant claim for current oral tobacco substitutes is to mimic the oral stimulation (oral fixation) effect of simply having something in the mouth to move around and suck on, with some attempt to simulate the typical flavorings found in oral tobacco products such as wintergreen or mint, but with no attempt to match the flavor or the salivation and stimulant effects of tobacco itself.

Thus, there is a need for oral stimulatory products for both in the alleviation of symptoms of oral conditions and as replacements for oral tobacco products.

### SUMMARY OF THE INVENTION

The invention provides an oral stimulatory product comprising a stimulating amount of at least one oral stimulant, the oral stimulant component being selected from the group consisting of Spilanthes, Szechuan Pepper, roasted Eucommia leaf, and a combination of Spilanthes, Szechuan Pepper, and/or roasted Eucommia leaf.

In at least some of its forms, the oral stimulatory product can act as an oral tobacco substitute, and as such, provides a number of advantages over the currently available retail oral tobacco alternate products. As an oral tobacco substitute, the oral stimulatory product of this invention closely simulates oral tobacco products in a manner that is not provided by currently available oral tobacco substitutes, including the oral sensation of burning, tingling, pepperiness and numbing that are experienced when actual oral tobacco products are used.

When the oral stimulatory product of the invention is a dosage form such as a lozenge, it provides at least some properties of an oral tobacco substitute, and also promotes salivation and/or alleviates the symptoms of mouth sores. The forms of the oral stimulatory product that are oral tobacco substitutes also promote salivation and may have a positive effect on mouth sores. The inventive oral stimulatory product does not provide any of the addictive or disease causing elements that are present in actual oral tobacco products.

### FURTHER DETAILED DESCRIPTION OF THE INVENTION

The oral stimulant component of the oral stimulatory product is a stimulating amount of at least one organic, oral stimulant, and is preferably comprised of Spilanthes (Acmella oleracea, also known as Spilanthes oleracea and Spilanthes acmella); Szechuan Pepper, (Zanthoxylum species, most commonly Z. piperitum, Z. simulans, and Z. schinifolium); ground, roasted Eucommia leaf (Eucommia ulmoides), or a combination of any two or more of the foregoing. Spilanthes, Szechuan Pepper, and roasted Eucommia leaf can be in the form of a finely ground herb, if desired. The Spilanthes and/or Szechuan Pepper stimulants provide a numbing, tingling and burning effect that may be considered similar to that of oral tobacco, and, along with roasted Eucommia leaf, stimulate salivation. Optionally and preferably, the oral stimulant component further comprises Black Pepper (Piper nigrum), which if desired can be in the form of a finely ground herb.

Spilanthes can be obtained commercially from, e.g., Robertet Inc. as Jambu (Spilanthes acmella) extract 15-40% in ethanol. Dried Spilanthes flower heads are obtained from local or global suppliers. Szechuan Pepper can be obtained commercially from, e.g., TeaWolf Inc. as an extract in ethanol. Dried Szechuan Peppers are obtained commercially from, e.g., Whole Herb Company or global suppliers. Black Pepper (Aquaresin® or oleoresin) can be obtained commercially from, e.g., Kalsec Inc. or other suppliers of Black Pepper extracts. Dried Black Peppers are obtained from local or global suppliers.

In addition to promoting salivation, roasted Eucommia leaf is added to the product to promote oral healing and for its anti-inflammatory and immune boosting effects. Roasted Eucommia leaf is commercially obtained from, e.g., Whole Herb Company or global suppliers.

All of the ingredients that are to be used in powdered form are milled to a fine powder. The ingredients to be used in powdered formed can be milled all together, or they can be milled individually or in any sub-combination and then mixed together. In an example of powdered ingredients, Spilanthes flower heads are finely milled. Similarly, if Szechuan Peppers are to be used, they are finely milled. In the same manner, Black Peppercorns are finely milled if they are to be used in powdered form.

Optional ingredients include Guarana extract, Yerba mate (Ilex paraguariensis), salt, orange peel, and natural flavors. In some aspects of the invention, Guarana extract and Yerba mate are used as sources of natural caffeine and other stimulant phytochemicals.

The oral stimulatory product of this invention as an oral tobacco substitute can closely replicate the appearance and oral sensation of actual oral tobacco products.

When the oral stimulatory product of this invention is an oral tobacco substitute, it may be consumed as a ‘chew’ (loose leaf) or contained within pouches of varying sizes to simulate retail, oral tobacco products. The oral tobacco substitute products are designed to mimic most or all of the five main effects derived from real oral tobacco products as noted above. The oral tobacco substitute comprises a base component and a stimulating amount of an oral stimulant component, the oral stimulant component being selected from the group consisting of Spilanthes, Szechuan Pepper, roasted Eucommia leaf, and a combination of Spilanthes, Szechuan Pepper, and/or roasted Eucommia leaf

When in the form of a finely milled powder, Spilanthes are used in an amount of about 1 to about 5 wt % of the total weight of the oral tobacco substitute. Similarly, if Szechuan Peppers are to be used in finely milled form, they are used in an amount of about 1 to about 5 wt % of the total weight of the oral tobacco substitute. Also similarly, when Black Pepper is used and is in finely milled form, it is used in an amount of about 0.5 to about 3 wt % of the total weight of the oral tobacco substitute.

When present by itself in the oral tobacco substitute, Spilanthes is preferably in an amount in the range of about 0.5 wt % to about 4 wt % relative to the total weight of the oral tobacco substitute. The Szechuan Pepper is preferably in an amount in the range of about 2 wt % to about 6 wt % relative to the total weight of the oral tobacco substitute when present by itself in the oral tobacco substitute. When both Spilanthes and Szechuan Pepper are present in the oral tobacco substitute, their combined amount is preferably about 5.5 wt %, and they can be present in any amount relative to each other. In preferred embodiments, the two ingredients are present in a ratio of 9:14 by weight of Spilanthes to Szechuan Pepper. Roasted Eucommia leaf is preferably present in an amount in the range of about 5 wt % to about 15 wt %; in preferred embodiments, the amount of roasted Eucommia leaf is about 6 wt %.

The oral stimulant component in the oral tobacco substitute formulation simulates the oral sensations common to tobacco based oral products. Black Pepper, when present in the oral tobacco substitute, simulates the burning and pepperiness of oral tobacco. Preferably, Black Pepper is present in the oral tobacco substitute in an amount in the range of about 0.3 wt % to about 1.5 wt % relative to the total weight of the oral tobacco substitute.

In combination, these four ingredients (Spilanthes, Szechuan Pepper, roasted Eucommia leaf, and Black Pepper) closely mimic the oral sensation provided by oral tobacco products when present in the preferred ranges indicated above. In addition, the four ingredients stimulate salivation in a manner very similar to that provided by oral tobacco products.

The material that provides the base component when the oral stimulatory product is an oral tobacco substitute comprises a cellulosic material that is safe for oral consumption. Suitable cellulosic materials include wood pulp, herbaceous plant leaves, such as mint leaves, corn silk, green tea leaves, and black tea leaves, preferably black tea leaves (Camellia sinensis). When tea leaves are used, carefully selecting the type and cut of the tea leaves provides a similar physical appearance and mouth feel to oral tobacco products. When black tea leaves are used in forming the oral tobacco substitute, they provide saliva (‘spit’) that is similar in appearance to that found when using actual oral tobacco products. Black tea has the added advantage of coloring the saliva in a manner similar to oral tobacco. However, by itself black tea does not provide the requisite oral sensation or the stimulant effect of oral tobacco.

Use of optional stimulants (e.g., Yerba mate and/or Guarana extract) in the oral tobacco substitute of this invention can simulate the stimulation provided by actual oral tobacco products.

Optionally, flavors can be included in the oral tobacco substitute. The inclusion of flavors found in actual oral tobacco products will simulate the flavor of actual tobacco products in the oral tobacco substitute. Preferably, natural flavors are employed to increase the tobacco flavor perception and to add typifying Wintergreen, Mint and other notes that are customarily found in oral tobacco products.

In some embodiments, to form the oral stimulatory product, especially an oral tobacco substitute, the base component is soaked in a liquid mixture comprised of at least one sweetener, an aqueous base solution, and a preservative. The liquid mixture contributes sweetness and flavor to the product. Preferably, the liquid mixture is comprised of molasses, glycerine, aqueous sodium hydroxide, and optionally but preferably aqueous sodium benzoate. The proportions of the liquid components in the liquid mixture and the relative amount of the liquid mixture used to infuse the base component affect the simulation of the texture, mouth feel and ‘pinchability’ (ease of removal of a ‘pinch’ of the product from the retail container and lack of wet or stained fingers) of the oral tobacco substitute. Optionally, a preservative, preferably sodium benzoate, is added to prevent spoilage of the finished product. The aqueous base raises the pH of the product to an alkaline value, which helps to soften the base component and promotes increased uptake of stimulating ingredients into the base component.

The proportions of individual liquid ingredients to each other and the relative amount of the liquid mixture to the base component affect the water activity (A_{w} or a_{w}) in the finished product, which in turn can help to prevent spoilage of the oral tobacco substitute by microorganisms such as bacteria, yeasts and molds.

The liquid mixture (e.g., the molasses, glycerine, aqueous sodium hydroxide and optional aqueous sodium benzoate) is blended and heated to a temperature between about 150 to about 195° F. (66 to 91° C.). Optionally, one or more natural flavors are blended with the liquid mixture. If liquid extracts (rather than solid particulates, powders, shavings or cuttings) of Spilanthes and/or Szechuan Pepper, optionally with roasted Eucommia leaf and Black Pepper, are to be used in the particular formulation, they are added at this stage.

The heated liquid mixture is added to the base component in a weight proportion of about 2:1 to about 5:1. The base component is then infused for about 15 minutes to about 12 hours. When the base component has become sufficiently softened and moistened, the excess liquid is removed by a mechanical method such as pressing, centrifugation, decantation, or filtration. The liquid mixture may be reused in a subsequent batch of product as a portion of a refreshed batch of liquid mixture typically comprising between about 80 wt % and about 90 wt % fresh liquid ingredients. The finely milled powder portion of the formulation is blended thoroughly with the infused tea leaves.

As an example of a formulation, an extract of Spilanthes containing 30% Spilanthol in ethanol is obtained commercially (Robertet Inc.), and is preferably used in an amount of about 0.05 to about 0.5 wt % relative to the total weight of the formulation. By extrapolation, other dilutions of Spilanthol extract in ethanol can be used at similar levels in the preparation of the oral tobacco substitute to achieve similar oral effects. An extract of Szechuan Pepper in ethanol (TeaWolf Inc.) is preferably used in an amount of about 0.02 to about 0.3 wt % relative to the total weight of the formulation. Szechuan Pepper extracts of other concentrations can be used to achieve similar levels in the liquid mixture by extrapolation. An extract of Black Pepper (Aquaresin®; Kalsec Inc.) is preferably used in an amount of about 0.1 to about 0.5 % relative to the total weight of the formulation. Again, similar amounts of Black Pepper can be used from extracts of other concentrations via extrapolation.

The finished oral tobacco substitute product is packed into suitable containers for retail sale.

Alternatively, the finished product is dispensed into suitable, porous, paper pouches for use as an alternate to oral tobacco pouches. In this embodiment of the invention, once the pouches have been packed and sealed, they are optionally sprayed with a portion of a liquid mixture that is the same as or similar to that used to soak the base component of the formulation. The of liquid mixture to the exterior of the pouch moistens it and ensures swift delivery of desirable oral sensation when the pouch is placed in the mouth, such as stimulation of salivation, accelerated oral delivery of stimulating and sensate components, and oral comfort.

A further use for the oral stimulatory product of this invention is to promote salivation (e.g., in relieving ‘dry mouth’), and/or to alleviate the symptoms of mouth sores (e.g., by numbing the pain from mouth ulcers, such as may be found in patients undergoing radiation and chemotherapy for cancer treatment and other conditions that tend to ulceration of the mouth). Preferably, to promote salivation or to alleviate the symptoms of mouth sores, the oral stimulatory product is a dosage form, such as a lozenge. Other dosage forms include capsules, tablets, and the like.

For the dosage forms, especially lozenges, Slippery Elm (Ulmus fulva) powder and a tableting agent are preferably included in the formulation. Tableting agents include those known in the art, and include calcium carbonate, magnesium stearate, microcrystalline cellulose, silicified microcrystalline cellulose, and the like. At least for forming lozenges, calcium carbonate and magnesium stearate are preferred tableting agents.

The active ingredients of the oral stimulatory product in lozenges are one or more oral stimulant components as described above. Preferably, all three of the ingredients described above (Spilanthes, Szechuan Pepper, and roasted Eucommia leaf) are included. The Spilanthes and/or Szechuan Pepper can be in the form of extracts.

Optional but preferred ingredients include Black Pepper, at least one sweetener such as stevia extract powder, and various flavors such as mint, vanilla, cherry, etc., to make the lozenge or other dosage form palatable.

A preferred dosage formulation for both promoting salivation and alleviating the symptoms of mouth sores, especially when the dosage form is a lozenge, includes Slippery Elm powder, a tableting agent, Spilanthes, roasted Eucommia leaf powder, and Szechuan Pepper.

In preferred dosage forms, Slippery Elm powder is in an amount of about 10 to about 30 wt %, preferably about 15 to about 20 wt %; a tableting agent is in an amount of about 40 to about 70 wt %, preferably about 50 to about 60 wt %; roasted Eucommia leaf powder is in an amount of about 10 to about 25 wt %, preferably about 15 to about 20 wt %; Szechuan Pepper is in an amount of about 0.3 to about 0.8 wt %, preferably about 0.4 to about 0.6 wt %, with all amounts relative to the total weight of the dosage form. When the dosage form is for promoting salivation, the Spilanthes is preferably in an amount of about 0.15 to about 0.45 wt %, more preferably about 0.25 to about 0.35 wt %; when the dosage form is for alleviating the symptoms of mouth sores, the Spilanthes is preferably in an amount of about 0.4 to about 1.5 wt %, more preferably about 0.9 to 1.3 wt %, again relative to the total weight of the dosage form. Also preferred in these dosage forms is the inclusion of stevia extract powder and a flavor.

To make the lozenges or other dosage forms, the components (oral stimulatory component(s), Slippery Elm powder, tableting agent(s), and optional ingredients, if any) are dry-blended, typically at room temperature, to form a dry-blended mixture; the dry-blended mixture is subjected to methods known in the art for forming the particular dosage form. e.g., extrusion, granulation and/or compression.

Another for the oral stimulatory product of this invention is use in a stepped nicotine addiction withdrawal program by formulating the inventive oral stimulatory with varying (decreasing) amounts of nicotine. Nicotine can be included in formulations for both the oral tobacco substitute products and the lozenges or other dosage forms.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation in its practice. Therefore the foregoing description is not intended to limit, and should not be construed as limiting, the invention to the particular exemplifications presented hereinabove.

1. An oral stimulatory product comprising a stimulating amount of at least one oral stimulant, the oral stimulant being selected from the group consisting of Spilanthes, Szechuan Pepper, roasted Eucommia leaf, and a combination of any two or all three of Spilanthes, Szechuan Pepper, and roasted Eucommia leaf.

2. An oral stimulatory product as in claim 1 wherein the oral stimulant further comprises Black Pepper.

3. An oral stimulatory product as in claim 1 optionally containing Guarana extract, Yerba mate, salt, orange peel, and/or natural flavors.

4. An oral stimulatory product as in claim 1 which is an oral tobacco substitute comprising a base component and a stimulating amount of an oral stimulant component, the oral stimulant component being selected from the group consisting of Spilanthes, Szechuan Pepper, roasted Eucommia leaf, and a combination of any two or all three of Spilanthes, Szechuan Pepper, and roasted Eucommia leaf.

5. An oral stimulatory product as in claim 4 wherein
Spilanthes is in an amount in the range of about 0.5 wt % to about 4 wt % relative to the total weight of the oral stimulatory product when Spilanthes is the only oral stimulant component;
Szechuan Pepper is in an amount in the range of about 2 wt % to about 6 wt % relative to the total weight of the oral stimulatory product when Szechuan Pepper is the only oral stimulant component;
Spilanthes and Szechuan Pepper are in a combined amount of about 5.5 wt % when both Spilanthes and Szechuan Pepper are present as oral stimulant components; and
roasted Eucommia leaf is in an amount in the range of about 5 wt % to about 15 wt % relative to the total weight of the oral stimulatory product when present in the oral stimulatory product.

6. An oral stimulatory product as in claims 5 wherein Black Pepper is present.

7. An oral stimulatory product as in claim 6 wherein the Black Pepper is in an amount in the range of about 0.3 wt % to about 1.5 wt % relative to the total weight of the oral tobacco substitute.

8. An oral stimulatory product as in claim 4 wherein Yerba mate and/or Guarana extract are included.

9. An oral stimulatory product as in claim 4 wherein the base component comprises a cellulosic material that is safe for oral consumption, which cellulosic material is selected from wood pulp and herbaceous plant leaves.

10. An oral stimulatory product as in claim 9 wherein the herbaceous plant leaves are mint leaves, corn silk, green tea leaves, and/or black tea leaves.

11. A method for forming an oral stimulatory product which is an oral tobacco substitute comprising an oral stimulant ingredient selected from the group consisting of Spilanthes, Szechuan Pepper, roasted Eucommia leaf, or a combination of any two or all three of Spilanthes, Szechuan Pepper, and roasted Eucommia leaf, the method comprising
a) forming a liquid mixture comprised of at least one sweetener, an aqueous base solution, and any components that are liquid extracts, and heating the liquid mixture to a temperature between about 150 to about 195° F.;
b) soaking the base component in the liquid mixture;
c) removing excess liquid; and
d) introducing any components that are in powder form.

12. A method as in claim 11 wherein
Spilanthes is in an amount in the range of about 0.5 wt % to about 4 wt % relative to the total weight of the oral stimulatory product when Spilanthes is the only oral stimulant component;
Szechuan Pepper is in an amount in the range of about 2 wt % to about 6 wt % relative to the total weight of the oral stimulatory product when Szechuan Pepper is the only oral stimulant component;
Spilanthes and Szechuan Pepper are in a combined amount of about 5.5 wt % when both Spilanthes and Szechuan Pepper are present as oral stimulant components; and
roasted Eucommia leaf is in an amount in the range of about 5 wt % to about 15 wt % relative to the total weight of the oral stimulatory product when present in the oral stimulatory product.

13. A method as in claim 12 wherein
Black Pepper is included;
Yerba mate and/or Guarana extract are included; and/or
the base component comprises a cellulosic material that is safe for oral consumption, which cellulosic material is selected from wood pulp and herbaceous plant leaves.

14. A method as in claim 13 wherein the Black Pepper is in an amount in the range of about 0.3 wt % to about 1.5 wt % relative to the total weight of the oral tobacco substitute.

15. An oral stimulatory product as in claim 1 wherein Spilanthes, Szechuan Pepper, and roasted Eucommia leaf are present.

16. An oral stimulatory product as in claim 1 which is a dosage form, and contains Slippery Elm powder and a tableting agent.

17. An oral stimulatory product as in claim 15 wherein at least one sweetener and/or a flavor are included.

18. An oral stimulatory product as in claim 17 wherein at least one sweetener is included, and wherein the sweetener is stevia extract powder.

19. An oral stimulatory product as in claim 16 wherein the tableting agent is calcium carbonate and/or magnesium stearate.

20. An oral stimulatory product as in claim 16 wherein
Slippery Elm powder is in an amount of about 10 to about 30 wt %;
the tableting agent is in an amount of about 40 to about 70 wt %;
roasted Eucommia leaf powder is in an amount of about 10 to about 25 wt %; and
Szechuan Pepper is in an amount of about 0.3 to about 0.8 wt %;
where the amounts are relative to the total weight of the dosage form.

21. An oral stimulatory product as in claim 20 wherein
the Spilanthes is in an amount of about 0.15 to about 0.45 wt %; or
the Spilanthes is in an amount of about 0.35 to about 0.65 wt %;
where the amounts are relative to the total weight of the dosage form.

22. A method for preparing an oral stimulatory product of claim 16, which method comprises
dry-blending the Slippery Elm powder, tableting agent, and at least one oral stimulant, to form a dry-blended mixture; and
forming the dry-blended mixture into a dosage form.

23. A method as in claim 22 wherein
Spilanthes, Szechuan Pepper, and roasted Eucommia leaf are present;
Black Pepper is present; and/or
the tableting agent is calcium carbonate and/or magnesium stearate.

24. A method as in claim 23 wherein
Slippery Elm powder is in an amount of about 10 to about 30 wt %;
the tableting agent is in an amount of about 40 to about 70 wt %;
roasted Eucommia leaf powder is in an amount of about 10 to about 25 wt %; and
Szechuan Pepper is in an amount of about 0.3 to about 0.8 wt %;
where the amounts are relative to the total weight of the dry-blended mixture.

25. A method as in claim 24 wherein
the Spilanthes is in an amount of about 0.25 to about 0.35 wt %; or
the Spilanthes is in an amount of about 0.4 to about 1.5 wt %;
where the amounts are relative to the total weight of the dry-blended mixture.

26. An oral stimulatory product of claim 1 in which nicotine is included.

27. A method as in claim 11 wherein nicotine is included.
